(19) United States
(12) Patent Application Publication
(10) Pub. No.: US 2023/0167393 A1
(43) Pub. Date: Jun. 1, 2023

(54) BIOREACTOR FOR CELL PROCESSING

(71) Applicant: Oribiotech LTD, London, Greater London (GB)

(72) Inventors: Farlan Veraitch, London, Greater London (GB); Jason Palmer, Yaxley Cambridgeshire (GB); Vincent Gowler, Cambridgeshire (GB); Tody Rickett, Cambridge, Cambridgeshire (GB); William Raimes, London, Greater London (GB); Arman Amini, London, Greater London (GB)

(21) Appl. No.: 17/905,999

(22) PCT Filed: Mar. 9, 2021
(86) PCT No.: PCT/GB2021/050578

§ 371 Date: Sep. 9, 2022

(30) Foreign Application Priority Data
Mar. 9, 2020 (GB) 2003406.2

(51) Int. Cl. C12M 1/00; C12M 1/34

(52) U.S. Cl. CPC C12M 23/22; C12M 1/34; C12M 23/26; C12M 41/44

(57) ABSTRACT

The present disclosure provides a bioreactor for cell processing. The bioreactor comprises a container including a base section comprising a sensor window chosen from a transparent sensor window or a translucent sensor window, a top section arranged opposite to the base section and comprising a fluid inlet and a fluid outlet, and a sidewall extending between the base section and the top section and defining an internal volume of the container adapted to hold a cell suspension. At least one optical element disposed on the sensor window within the internal volume, the at least one optical element adapted to emit a fluorescence signal in response to incident light, the fluorescence signal associated with one or more parameters of the cell suspension.

### CROSS-REFERENCE TO RELATED APPLICATIONS

This application is a national phase entry under 35 U.S.C. § 371 of International Patent Application PCT/GB2021/050578, entitled “A BIOREACTOR FOR CELL PROCESSING,” filed Mar. 9, 2021, designating the United States of America and published as International Patent Publication WO 2021/181078 A1 on Sep. 16, 2021, which claims the benefit under Article 8 of the Patent Cooperation Treaty to Great Britain Patent Application Serial No. 2003406.2, filed Mar. 9, 2020.

### TECHNICAL FIELD

This disclosure relates to a bioreactor for cell processing. In particular, this disclosure relates to a bioreactor for cell processing having an optical element.

### BACKGROUND

Cell and gene therapy manufacturing processes are often complex and include manual or semi-automated steps across several devices. Equipment systems used in various steps (i.e., unit operations) of cell-based therapeutic products (CTP) manufacturing may include devices for cell collection, cell isolation/selection, cell expansion, cell washing and volume reduction, cell storage and transportation. The unit operations can vary immensely based on the manufacturing model (i.e., autologous versus allogenic), cell type, intended purpose, among other factors. In addition, cells are “living” entities sensitive to even the simplest manipulations (such as differences in a cell transferring procedure). The role of cell manufacturing equipment in ensuring scalability and reproducibility is an important factor for cell and gene therapy manufacturing.

In addition, cell-based therapeutic products (CTP) have gained significant momentum thus there is a need for improved cell manufacturing equipment for various cell manufacturing procedures, for example but not limited to stem cell enrichment, generation of chimeric antigen receptor (CAR) T cells, and various cell manufacturing processes such as collection, purification, gene modification, incubation/recovery, washing, infusion into patient and/or freezing.

The culture or processing of cells typically requires the use of a device to hold the cells, for example in an appropriate culture medium when culturing the cells. The known devices include shaker flasks, roller bottles, T-flasks and bags. Such bottles or flasks are widely used but suffer from several drawbacks. Chief among the problems are the requirement for transfer of cells without contamination when passaging or processing subsequently and the sterile addition of supplements and factors. The existing cell culture devices require re-supply of culture medium and oxygen for continued cell growth. Gas permeable cell culture devices are described in U.S. Pat. No. 8,415,144. However, such devices also require transfer of medium and/or cells in and out of the devices.

A key limiting factor in the production of cells or gene therapies for use in medicine is the absence of compact, automated closed systems for performing unit operations without contamination. For example during cell culture, upstream or subsequent processing of cells, there is a risk of contamination when making additions to the culture vessel, or when removing cells or removing liquid samples. The operating systems are largely manual and hence expensive to operate. Multiple pieces of equipment are typically required to cover all of the non-cell culture steps, which involves many transfers, each of which is an opportunity for operator errors and contamination to occur. Furthermore with increasing manual operations comes increasing risk of manual errors and therefore the current labor-intensive processes lack the robustness required for the manufacture of clinical-grade therapeutics.

There is therefore a need for cell processing devices (e.g., multistep cell processors) which permit such processing which avoids the requirement for constant movement of cells into fresh devices. For example, it would be advantageous if scale-up of cells in culture could be achieved without transfer of cells into a larger device as the cell population for any given culture increases.

Previous cell manufacturing devices use complex equipment which is large and difficult to assemble. The devices use complex networks of tubing, valves and pumps to link elements of the equipment together.

### BRIEF SUMMARY

It is an object of certain aspects of the present disclosure to provide an improvement over the above described techniques and known art; particularly to provide a bioreactor and systems that facilitate the monitoring of cells.

In accordance with a first aspect of the present disclosure there is provided a bioreactor for cell processing. The bioreactor comprises a container having a base section comprising a transparent or translucent sensor window, a top section arranged opposite to the base section and comprising a fluid inlet and a fluid outlet, and a sidewall extending between the base section and the top section and defining an internal volume of the container adapted to hold a cell suspension. At least one optical element disposed on the sensor window within the internal volume, the optical element being adapted to emit a fluorescence signal in response to incident light, the fluorescence signal being associated with one or more parameters of the cell suspension.

In accordance with a second aspect of the present disclosure, there is provided a cell processing system. The cell processing system comprises a bioreactor comprising a container having a base section comprising a transparent or translucent sensor window, a top section arranged opposite to the base section and comprising a fluid inlet and a fluid outlet, and a sidewall extending between the base section and the top section and defining an internal volume of the container adapted to hold a cell suspension. At least one optical element disposed on the sensor window within the internal volume, the optical element being adapted to emit a fluorescence signal in response to incident light, the fluorescence signal being associated with one or more parameters of the cell suspension. An optical sensor is positioned proximate to an outer surface of the sensor window to sense a fluorescence signal emitted by the optical element associated with one or more parameters of the cell suspension. A controller is configured to receive a sensor signal from the optical sensor, the sensor signal corresponding to the one or more parameters of the cell suspension.

In accordance with a third aspect of the present disclosure, there is provided a method of cell processing. The method comprises providing a cell processing system. The cell processing system comprises a bioreactor comprising a container having a base section comprising a transparent or translucent sensor window, a top section arranged opposite to the base section and comprising a fluid inlet and a fluid outlet, and a sidewall extending between the base section and the top section and defining an internal volume of the container adapted to hold a cell suspension. At least one optical element disposed on the sensor window within the internal volume, the optical element being adapted to emit a fluorescence signal in response to incident light, the fluorescence signal being associated with one or more parameters of the cell suspension. An optical sensor is positioned proximate to an outer surface of the sensor window to sense a fluorescence signal emitted by the optical element associated with one or more parameters of the cell suspension. A controller is configured to receive a sensor signal from the optical sensor, the sensor signal corresponding to the one or more parameters of the cell suspension. The method further comprises sensing a fluorescence signal emitted by the optical element associated with the one or more parameters of the cell suspension using the optical sensor.

In accordance with a fourth aspect of the present disclosure there is provided a bioreactor for cell processing. The bioreactor comprises a container having a base section, a top section arranged opposite to the base section and comprising a fluid inlet and a fluid outlet, and a sidewall extending between the base section and the top section and defining an internal volume of the container adapted to hold a cell suspension. The bioreactor further comprises at least one chemical sensor disposed on the base section within the internal volume for sensing one or more parameters of the cell suspension.

Suitably, such bioreactors, sensor arrangements, cell processing systems and cell processing methods are suitable for automated cell processing methods, and allows for continuous or periodic monitoring of parameters of the cell suspension.

The bioreactor may further comprise at least one optical sensor positioned proximate to an outer surface of the sensor window. This provides for non-invasive sensing of parameters of the cell suspension and therefore maintains a sterile environment while desired parameters of the cell suspension can be sensed. The optical sensor may be positioned at one end of an optical fiber cable. An opposite end of the optical fiber cable may be in alignment with the optical element. This provides for positioning the optical sensor away from the base of the bioreactor. The optical fiber cable may transfer LED light to the optical element and transfer the fluorescence signal emitted by the optical element to the optical sensor.

The optical sensor may be in alignment with the optical element.

The optical sensor may comprise an LED arranged to emit light onto the optical element. The optical sensor may be configured to receive the fluorescence signal emitted by the optical element. It will be understood that the fluorescence signal emitted by the optical element will be altered by absorption of energy from some of the excited molecules of the optical element by analytes of the cell suspension held in the container. Accordingly, the fluorescence signal received at the optical sensor corresponds to one or more parameters of the cell suspension. In particular, the one or more parameters of the cell suspension may be a dissolved oxygen concentration, and/or a pH, and/or a dissolved carbon dioxide concentration.

At least two optical elements may be disposed on the sensor window. Each optical element may have a corresponding optical sensor. Alternatively, one optical sensor may be movable to be aligned with different optical elements, so that one optical sensor can be used with a plurality of optical elements.

The at least one optical element may have a circular, or substantially circular, shape. The at least one optical element may have a kidney bean shape or a ring shape. Suitably, the kidney bean shape or the ring shape allows for the at least one optical sensor to remain in alignment with the at least one optical element during rotation of the bioreactor.

The at least one optical element may be positioned at or near a center position of the base section. This provides for the accurate measurement of a low volume of cell solution as low volumes of cell solution in the container will still cover the optical element.

The bioreactor may further comprise at least one chemical sensor. The at least one chemical sensor may be a glucose sensor and/or a lactate sensor. The at least one chemical sensor may be an enzymatic-based sensor.

The chemical sensor may include an electrode and a connecting wire. The connecting wire may extend through a slit in the base section of the bioreactor. The slit in the base section may be sealed about the connecting wire. The connecting wire may extend through an opening in the top section of the bioreactor.

The bioreactor may further comprise a temperature sensor. A temperature detected by the temperature sensor may be used to compensate for thermal drift in the optical sensors.

The bioreactor may further comprise a sensor selected from one or more of a pressure sensor, a flow sensor, an accelerometer, a capacitance sensor, an ammonia sensor, an optical sensor and/or a camera.

The sidewall of the container may comprise a compressible wall element. The compressible wall element may have a bellows arrangement. The base section may be engageable by, or connectable to, an agitator operable to move the base section relative to the top section to compress or extend the compressible wall. This provides for compression and extension of the container to stimulate mixing of the contents of the bioreactor, and further provides for controlled agitation, including compression and extension, of the bioreactor.

In examples, the transparent or translucent sensor window may be substantially planar (i.e., flat). In other examples the transparent or translucent sensor window may be sloped or frustoconical. The transparent or translucent sensor window may be positioned at a bottom-most point of the container. This provides for the accurate measurement of a low volume of cell solution.

The base section and/or the top section may be substantially circular, providing a substantially cylindrical bioreactor. The base section and/or the top section may alternatively be any suitable shape, for example square, triangular, ovular, or any polygonal shape.

The cell processing system may further comprise an agitator arranged to engage the base section of the bioreactor. The agitator may be operable to move the base section relative to the top section to compress or extend the compressible wall.

The agitator may comprise an agitation plate arranged to engage the base section of the container. The agitation plate may have an aperture for receiving the optical sensor. Suitably, this an arrangement holds the optical sensors in alignment with the optical elements on the base section of the container.

The sidewall of the container may comprise a compressible wall element. The controller may be configured to control the agitator to move the base section relative to the top section to stimulate mixing of a fluid within the bioreactor. This provides for control of the mixing of the fluid in the bioreactor, which can increase the levels of dissolved oxygen in the cell suspension.

The controller may be configured to adjust a condition within the bioreactor based on the received fluorescence signal. The controller may be configured to adjust the condition within the bioreactor until the parameter is equal to a target parameter. This provides for automated control of the bioreactor based on parameters sensed by the one or more sensors of the bioreactor.

The controller may adjust the condition within the bioreactor by adjusting the gas flow into the bioreactor. This may provide for control of the gas concentration in the bioreactor.

The method may further comprise adjusting a condition within the bioreactor based on the sensed fluorescence signal.

Adjusting the condition within the bioreactor may include adjusting the gas flow into the bioreactor. This provides for control of the gas concentration in the bioreactor.

The sidewall of the container may comprise a compressible wall element. Adjusting the condition within the bioreactor may include moving the base section relative to the top section to stimulate mixing of a fluid within the bioreactor. This provides for control of the mixing of the fluid in the bioreactor which can increase the levels of dissolved oxygen in the cell suspension.

The bioreactor may be adapted to hold a bioreactor fluid. The bioreactor fluid may comprise a cell suspension. Suitably the cell suspension comprises a population of cells present in a liquid medium.

Suitably the population of cells may comprise any cell type. Suitably the population of cells may comprise a homogenous population of cells. Alternatively the population of cells may comprise a mixed population of cells.

Suitably the population of cells may comprise any human or animal cell type, for example: any type of adult stem cell or primary cell, T cells, CAR-T cells, monocytes, leukocytes, erythrocytes, NK cells, gamma delta t cells, tumor infiltrating t cells, mesenchymal stem cells, embryonic stem cells, induced pluripotent stem cells, adipose derived stem cells, Chinese hamster ovary cells, NSO mouse myeloma cells, HELA cells, fibroblasts, HEK cells, insect cells, organoids, etc. Suitably the population of cells may comprise T-cells.

Alternatively, the population of cells may comprise any microorganism cell type, for example: bacterial, fungal, Archaean, protozoan, algal cells.

Suitably the liquid medium may be any sterile liquid capable of maintaining cells. Suitably the liquid medium may be selected from: saline or may be a cell culture medium. Suitably the liquid medium is a cell culture medium selected from any suitable medium, for example: DMEM, XVIVO 15, TexMACS. Suitably the liquid medium is appropriate for the type of cells present in the population. The skilled person is aware of suitable media to use when culturing cells.

For example, the population of cells comprises T cells and the liquid medium comprises XVIVO 10.

Suitably the liquid medium may further comprise additives, for example: growth factors, nutrients, buffers, minerals, stimulants, stabilizers or the like.

Suitably the liquid medium comprises growth factors such as cytokines and/or chemokines. Suitably the growth factors are appropriate for the type of cells present in the population and the desired process to be carried out. Suitably the liquid medium comprises stimulants such as antigens or antibodies, which may be mounted on a support. Suitable stimulants are appropriate for the type of cells present in the population and the desired process to be carried out. Suitably, when culturing T-cells, for example, antibodies are provided as a stimulant in the liquid medium. Suitably the antibodies are mounted on an inert support such as beads, for example: dynabeads.

Suitably the additives are present in the liquid medium at an effective concentration. An effective concentration can be determined by the skilled person on the basis of the population of cells and the desired process to be carried out using known teachings and techniques in the art.

Suitably the population of cells are seeded in the liquid medium at a concentration of between 1×10⁴ cfu/ml up to 1×10⁸ cfu/ml.

### BRIEF DESCRIPTION OF THE DRAWINGS

Embodiments of the disclosure are further described hereinafter with reference to the accompanying drawings, in which:

FIG. 1 illustrates a perspective view of a bioreactor according to a first embodiment of the disclosure.

FIG. 2 illustrates a perspective view of the bioreactor according to a second embodiment of the disclosure.

FIG. 3A illustrates a cross-sectional perspective view of the bioreactor according to the first embodiment.

FIG. 3B illustrates an enlarged cross-sectional perspective view of the base of the bioreactor according to the first embodiment.

FIG. 4 illustrates a cross-sectional perspective view of the bioreactor according to the first embodiment connected to an agitation plate.

FIG. 5 illustrates a close up view of the bioreactor according to the second embodiment connected to an agitation plate.

FIG. 6A illustrates a diagram of a side view of the bioreactor according to the second embodiment connected to the agitation plate in a lowered position.

FIG. 6B illustrates a diagram of a side view of the bioreactor according to the second embodiment connected to the agitation plate in a raised or agitation position.

FIG. 7 illustrates a cross-sectional perspective view of the bioreactor according to a third embodiment positioned within the cell processing unit prior to connecting the agitation plate.

FIG. 8 illustrates a perspective view of the bioreactor according to the third embodiment positioned within the cell processing unit prior to connecting the agitation plate.

FIG. 9 illustrates a perspective view of an agitation mechanism in the cell processing unit according to the second embodiment.

FIG. 10 illustrates a perspective view of the bioreactor and the agitation mechanism in the cell processing unit according to the third embodiment.

FIG. 11 illustrates a cross-sectional perspective view of the bioreactor and the agitation plate in the cell processing unit according to the third embodiment.

FIG. 12 illustrates a cross-section view of the bioreactor and the agitation plate according to the third embodiment.

FIG. 13A illustrates a cross-sectional perspective view of the bioreactor according to a fourth embodiment of the disclosure.

FIG. 13B illustrates a cross-sectional perspective view of the bioreactor according to a fifth embodiment of the disclosure.

FIG. 14 illustrates a cross-sectional perspective view of the bioreactor according to a sixth embodiment of the disclosure.

FIG. 15 illustrates a cross-sectional perspective view of the bioreactor according to a seventh embodiment of the disclosure.

### DETAILED DESCRIPTION

Specific embodiments of the disclosure will now be described with reference to the accompanying drawings. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art. The terminology used in the detailed description of the embodiments illustrated in the accompanying drawings is not intended to be limiting of the disclosure. In the drawings, like numbers refer to like elements.

The terminology used herein is for the purpose of describing particular aspects of the disclosure only, and is not intended to limit the disclosure. As used herein, the singular forms “a,” “an” and “the” are intended to include the plural forms as well, unless the context clearly indicates otherwise.

In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation, for example, definition of dimensions such as width or breadth or height or length or diameter depends on how exemplary aspects are depicted, hence, if depicted differently, a shown width or diameter in one depiction is a length or thickness in another depiction.

It should be noted that the words “comprising,” “having” or “including” do not necessarily exclude the presence of other elements or steps than those listed and the words “a” or “an” preceding an element do not exclude the presence of a plurality of such elements. It should further be noted that any reference signs do not limit the scope of the claims, that the example aspects may be implemented at least in part by way of both hardware and software, and that several “means,” “units” or “devices” may be represented by the same item of hardware.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

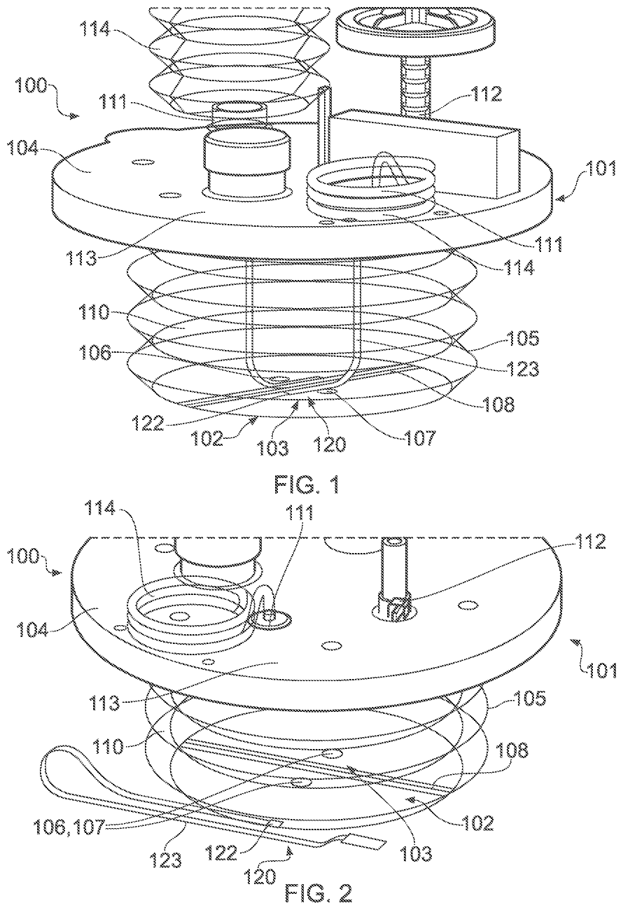

FIGS. 1 and 2 illustrate a bioreactor 100 including a container 110. The container has a top section 101, a base section 102 and a sidewall 105 extending between the base section 102 and the top section 101 and defining an internal volume of the container 110. The top section 101 is arranged opposite the base section 102. The top section 101 may be substantially parallel to the base section 102.

The base section 102 is planar, i.e., flat. However, the base section 102 may alternatively be sloped, and/or curved. In one example, the base section is frustoconical. The base section 102 may include a lower portion of a wall extending from the planar, slanted, frustoconical, or curved base, or the base section may include attached or integrally formed attachment features.

The base section 102 has a transparent, translucent sensor window 103. The sensor window 103 is non-opaque. In particular, the sensor window 103 is transparent or translucent to the light emitted by an LED of an optical sensor, and is transparent or translucent to the fluorescence emitted by an optical element 106, 107, as explained further hereinafter.

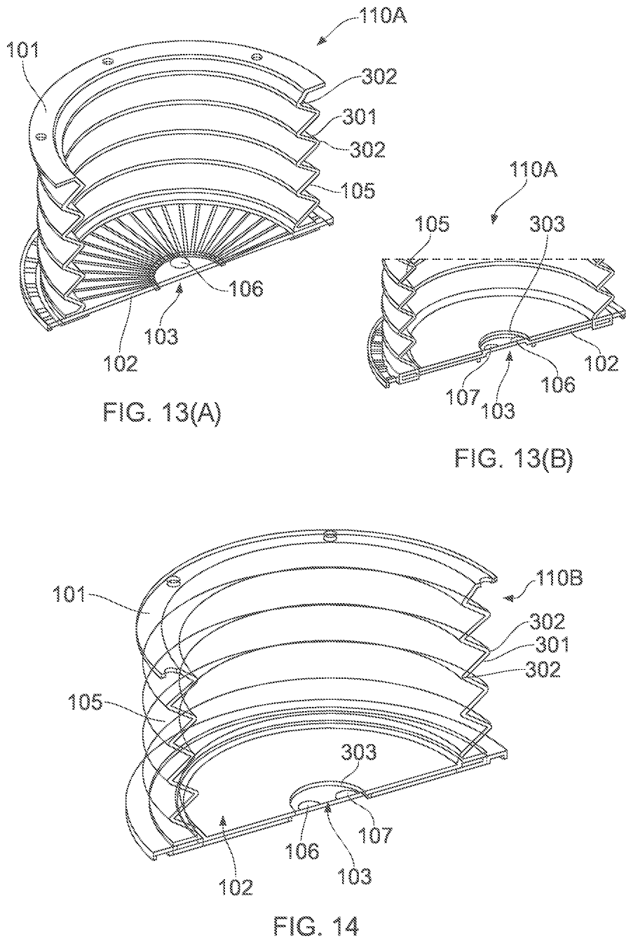

In the example illustrated in FIGS. 1 and 2, the entire base section 102 is formed from a transparent or translucent material, and a part of the base section 102 provides a sensor window 103. In another example, as illustrated in FIGS. 13B to 15, the sensor window 103 may be only a central portion of the base section 102 formed from the transparent or translucent material. In other examples, a non-central portion of the base section 102 formed from the transparent or translucent material. The base section 102 may be formed from an opaque material having a transparent or translucent material to form the sensor window 103. The transparent or translucent material may be integrally molded with the base section 102. The sensor window 103 may be planar (i.e., flat).

The top section 101 may include a cell processing platform 104. As used herein, the term “cell processing platform” is used to describe an interface that allows for various unit operations to be performed thereon. The cell processing platform may be an interface that permits the introduction of material to, or extraction of material from, one or more containers interfaced, or otherwise operably coupled to, the cell processing platform.

The cell processing platform 104 comprises a body having an upper surface and a lower surface. The cell processing platform 104 has a fluid inlet 111 and a fluid outlet 112. The cell processing platform 104 may have a plurality of fluid inlets and/or a plurality of fluid outlets. The body 113 of the cell processing platform 104 is shown as being generally circular and planar. The body 113 includes one or more resealable ports spaced radially about the body 113. The resealable ports extend from the upper surface, through the body 113, and to the lower surface. The resealable ports may comprise a septum seal.

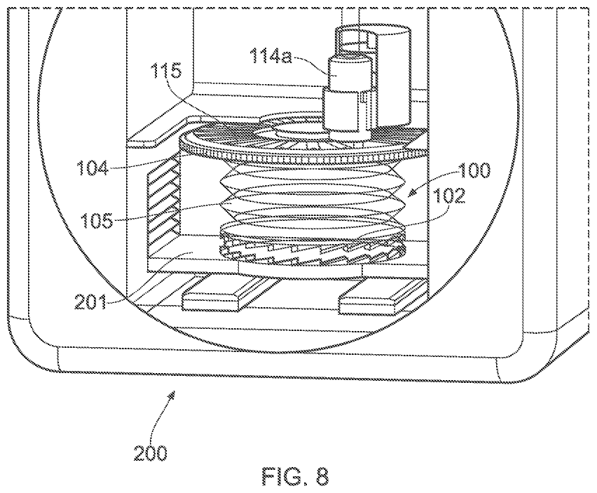

Accessories 114 can be mounted to the cell processing platform which can be actuated to pierce the at least one resealable port to provide a fluid pathway therethrough. The accessory 114 may be a sterile connector 114a (see FIG. 8) having a piercing element and, upon actuation, the sterile connector 114a may provide an aseptic fluid pathway from an accessory container attached to the sterile connector to the container 110 connected to the cell processing platform 104. The accessory container attached to the sterile connector 114a may hold cells, media, beads or viruses. The accessory container attached to the sterile connector 114a may facilitate sampling, waste extraction or cell extraction. The sterile connector 114a may be that described in patent application PCT/GB2020/053229.

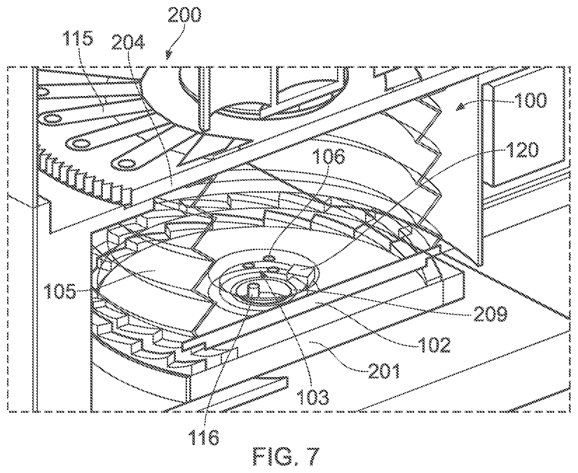

For example, upon input to a control panel, a controller of a cell processing system may cause a drive apparatus to actuate such that the cell processing platform 104 is caused to move, for example rotate about a central longitudinal axis. The controller may cause the cell processing platform 104 to move such that a particular resealable port of the cell processing platform 104 is caused to align with a piercing member of the sterile connector 114a so as to enable piercing of the resealable port upon actuation of the sterile connector 114a. Once coaxially aligned, the cell processing platform 104 may be raised or the accessory mounting member may be lowered, thereby causing face-to-face engagement of the accessory to a particular one or the at least one resealable ports. In this way, an aseptic paper seal 115 (see FIGS. 7 and 8) of the at least one resealable port is caused to mate with a corresponding aseptic paper seal of the accessory mounted to the accessory mounting member. The controller then causes actuation of a portion of the accessory mounting member to remove both aseptic paper seals, thereby providing an aseptic connection between the accessory and the at least one resealable port. The cell processing system then causes the desired operation of the accessory, examples of which are provided below.

The cell processing system may alternatively be manually controlled. The cell processing platform 104 may be rotated by a user to expose a particular resealable port. A sterile connector 114a may then be connected to the resealable port such that the aseptic paper seal 115 of the at least one resealable port is caused to mate with the corresponding aseptic paper seal of the accessory mounted to the accessory mounting member. A suitable accessory container can then be connected to the sterile connector 114a. The aseptic paper seal 115 can then be removed and the sterile connector 114a actuated to provide a fluid connection between the accessory container and the internal volume of the container 110.

As illustrated in FIGS. 1 and 2, the sidewall 105 is a flexible or a compressible wall. The compressible wall may take the form of a concertina or the like. The container 110 may be regarded as a bellows-based container. As explained further hereinafter with reference to FIGS. 13A to 15, the sidewall 105 may have a plurality of lateral rigid sections 301 arranged in parallel with the base section 102. Each pair of adjacent lateral rigid sections 301 is interleaved with a deformable region 302 so as to allow compression of the bioreactor along the longitudinal axis. The deformable regions 302 may be hinges that alternate inward and outward to provide collapsibility of the container 110. The hinges may be formed by thinning of the sidewall 105 material. Directionality of the hinges may be provided by thinning on either the inner or the outer side of the sidewall 105. The lateral rigid sections 301 and deformable regions 302 extend from the top section 101 of the container to the base section 102 allowing for complete compression of the container.

In the examples of FIGS. 1 and 2 the bioreactor 100 has two optical elements 106, 107 adhered to the sensor window 103 of the base section 102. The optical elements illustrated are optical dots 106, 107. The optical dots 106, 107 are positioned at a central portion of the base section 102. In this example, the entire base section 102 is transparent or translucent, so a portion of the base section 102 provides the sensor window 103. The base section 102 may comprise a seam, for example a central seam 108, formed during manufacture (e.g., due to molding). The optical dots 106, 107 may be positioned on opposite sides of the central seam 108 of the bioreactor 100.

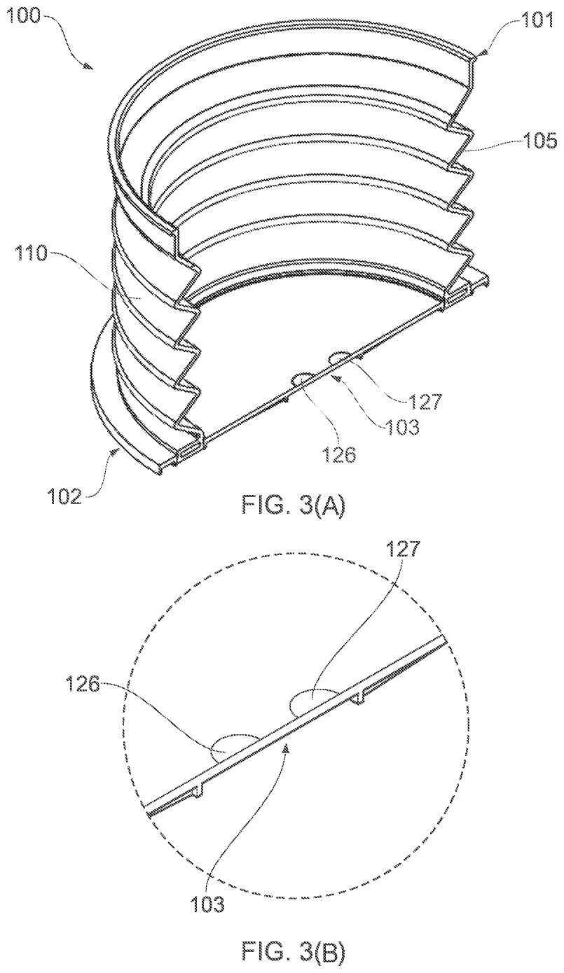

The optical dots 106, 107 have an embedded fluorescent dye. In combination with an optical sensor, the optical dots 106, 107 are used for the measurement of pH and dissolved oxygen of the cell suspension contained in the bioreactor. Optical dots may also be used for the measurement of dissolved carbon dioxide. The optical dots 106, 107 may be self-adherent, or alternatively any suitable adhesive may be used to attach the optical dot 106, 107 to the base section 102. The optical dots 106, 107 may alternatively be integrally formed in the sensor window. According to one example, the sensor window 103 may be overmolded onto the optical dots, or the sensor window 103 and the optical dots may be co-injected. According to another example, as illustrated in FIGS. 3A and 3B, the base section may have two recesses 126, 127 on an inner surface of the base section 102. Each recess may be sized to receive one of the optical dots 106 or 107. The optical dots 106, 107 may be adhered to an inner surface of the recesses 126, 127. The recess may allow for the optical dots 106, 107 to be secured to the base section 102 such that an upper surface of the optical dots 106, 107 is flush with the inner surface of the base section 102, thereby providing a smooth inner surface of the base section 102.

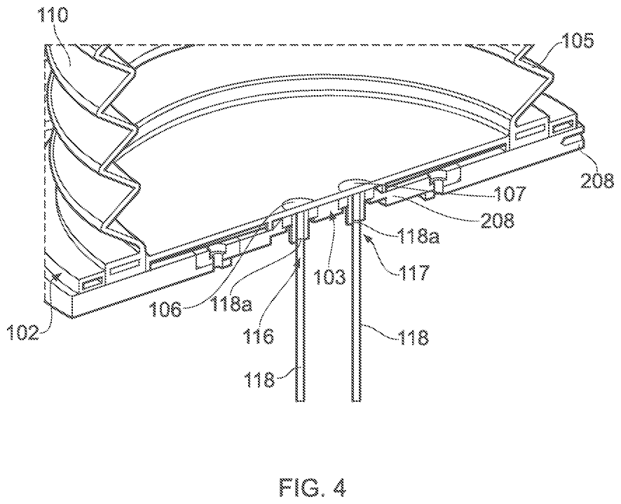

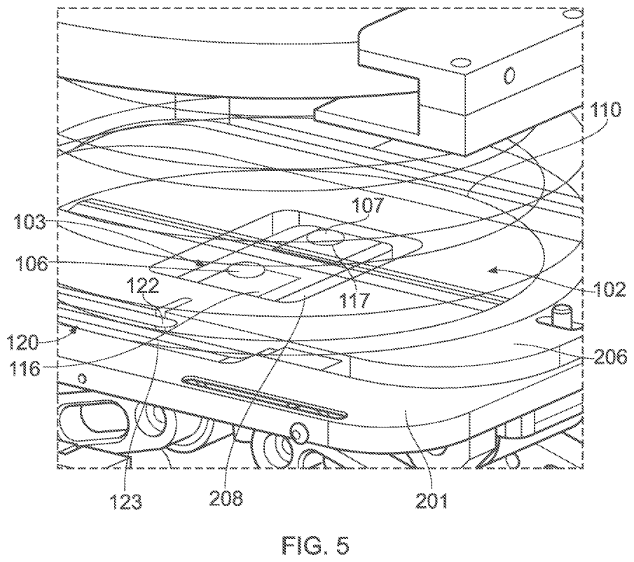

FIGS. 4 and 5 illustrate two optical sensors 116, 117 positioned near an outer surface of the sensor window 103 and in alignment with a corresponding optical dot 106, 107, respectively. The optical sensors 116, 117 are mounted on a part of a housing of the cell processing unit 200 of which the bioreactor 100 forms a part. For example, the optical sensors 116, 117 may be positioned on an optical sensor mounting block 208 mounted to an agitation plate 201 as described further hereinafter.

Alternatively, as illustrated in FIG. 7, a single optical sensor 116 may be utilized. The single optical sensor can be aligned with each optical dot in turn by relative movement of the base section 102 and the optical sensor 116.

The optical sensors 116, 117 may remain stationary so that in order to measure the parameters of the cell suspension, the optical dots 106, 107 and the optical sensors 116, 117 must be rotationally aligned. Alternatively, the optical sensors 116, 117 may be rotationally mounted in the cell processing unit 200. This allows the sensors 116, 117 to rotate in order to align with the optical dots 106, 107 as the bioreactor 100 is rotated.

Preferably, the optical sensors 116, 117 are positioned less than 5 millimeters from the base of the bioreactor, more preferably the optical sensors are positioned between 2 to 4 mm from the base of the bioreactor when an optical measurement is taken.

Each optical sensor 116, 117 includes an LED. Each optical sensor 116, 117 may include a reader. As illustrated in FIG. 4, each optical sensor may also include an optical fiber 118 to transfer light from the LED to an end of the optical fiber 118a in alignment with the optical dot 106, 107 and to transfer detected light back to the reader. The optical sensors 116, 117 are preferably concentrically aligned with the optical dots 106, 107. The optical sensors 116, 117 are non-invasive and do not require direct contact with the cell suspension in the bioreactor. In particular, as explained below, the optical sensors 116, 117 operate by emitting light through the transparent or translucent sensor window 103 and receiving fluorescence through the transparent or translucent sensor window.

To measure a parameter of the cell suspension, for example dissolved oxygen, pH or dissolved carbon dioxide, light from the LED in the optical sensor 116, 117 is directed toward the optical dot 106, 107. The light from the LED passes through the transparent or translucent sensor window 103 in the base section 102. Incident light causes excitation of the molecules in the fluorescent dye which causes the molecules to emit fluorescence in response. The energy from the excited molecules is absorbed by an analyte in contact with the optical dot, such as oxygen or carbon dioxide in the cell suspension, thereby quenching the fluorescence. The reader in the optical sensor measures a fluorescence signal passing through the transparent or translucent sensor window 103 in order to determine the quenching of the signal over time caused by the absorption of excited molecules by the analyte in the cell suspension. Therefore, the fluorescence signal is associated with the parameter of the cell suspension.

The optical dots 106, 107 illustrated in FIGS. 1 and 2 are circular. However, it will be appreciated that the optical dots (optical elements) 106, 107 may be any suitable shape, such as a triangular or square shape. The optical dots (optical elements) 106, 107 may have a kidney bean shape or may have a ring shape. This allows for the optical sensors 116, 117 to remain in alignment with the optical dots 106, 107 during rotation of the bioreactor 100.

The optical dots 106, 107 and optical sensors 116, 117 may be the preSens® optical oxygen, optical pH or optical carbon dioxide measurement systems.

FIGS. 1 and 2 further illustrate a bioreactor 100 having a chemical sensor 120 connected to the inner surface of the base section 102. The chemical sensor 120 includes an electrode 122 and a connecting wire 123. Each illustrated chemical sensor may be an individual glucose or lactate sensor or may be a combined glucose and lactate sensor. The bioreactor 100 may comprise a first chemical sensor for measuring glucose and a second chemical sensor for measuring lactate. The chemical sensor 120 may be an enzymatic-based sensor. The chemical sensor may be the CapSensors® from C-CIT Sensors AG. The chemical sensor 120 is adhered to the inner surface of the base section 102. Any suitable adhesive may be used. The adhesive preferably cures at room temperature. The chemical sensor 120 may be connected to a Bluetooth device which can send the sensor readings to a controller (not shown).

In the example of FIG. 1, the connecting wire 123 of each chemical sensor 120 extends through an opening in the top section 101 of the bioreactor 100. According to another second example as shown in FIG. 2, the connecting wire 123 of the chemical sensor 120 extends through the central seam 108 where the central seam 108 forms a slit in the base section 102 of the bioreactor. The slit can be sealed about the connecting wire 123 by any suitable adhesive to seal the base section 102 once the chemical sensor 120 has been inserted.

The bioreactor 100 illustrated in FIGS. 1 and 2 includes a combination of optical dots 106, 107 and chemical sensors 120. According to other examples, the bioreactor comprises only one of the optical dots or the chemical sensor.

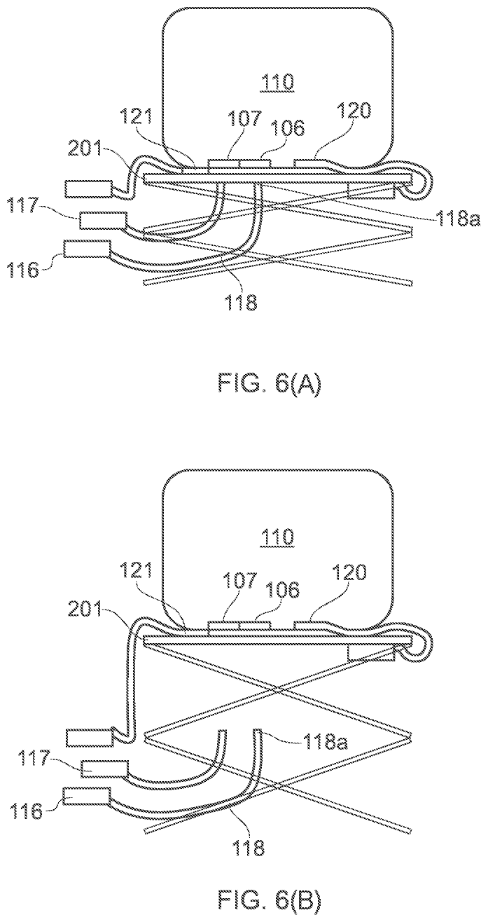

The bioreactor 100 may further include a temperature sensor 121, for example a thermocouple, as illustrated in FIGS. 6A and 6B. Measurement of the bioreactor temperature allows for adjustment of the data received from the optical sensor 116, 117 and/or the chemical sensor 120 to compensate for thermal drift in the optical sensors 116, 117 and/or chemical sensors 120 and thus improve sensor readings. The temperature sensor 121 may be positioned in contact with the cell suspension, on an outer surface of the bioreactor 100, or connected to the agitation plate 201 as will be discussed below.

Any other appropriate sensors are contemplated for use with the bioreactor. Examples of such sensors include, but are not limited to, pressure sensors, flow sensors, accelerometers, capacitance sensors, ammonia sensors, optical sensors, cameras, and the like. Examples of other parameters which may be sensed include, but are not limited to, optical density, light scattering, images of cells, metabolic turnover, rate of consumption by cells, capacitance, pressure, flow rate, movement of the bioreactor base, and the like.

The sensors described herein may be connected to, or integral to, any part of the bioreactor 100, such as the base section 102, the top section 101 and/or the sidewall 105.

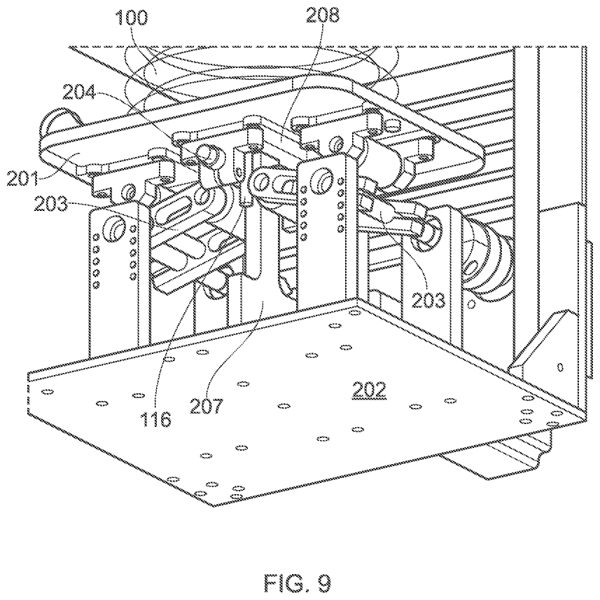

FIGS. 8 and 9 illustrate a cell processing system including the cell processing platform 104 and the bioreactor 100 loaded into a cell processing unit 200. The cell processing system is suitable for performing, or enabling, one or more unit operations of cell processing, for example cell and/or gene therapy manufacture. The cell processing system may be suitable for performing, or enabling, cell collection, cell isolation, cell selection, cell expansion, cell washing, volume reduction or wasting, cell storage or cell transportation. The cell processing unit 200 is a housing for enclosing components of the cell processing system as described herein. The cell processing unit 200 may take the form of an incubator or the like. The cell processing unit 200 may provide a controlled environment, including temperature, carbon dioxide concentration and/or oxygen concentration, therein that is suitable for performing one or more unit operations in cell processing.

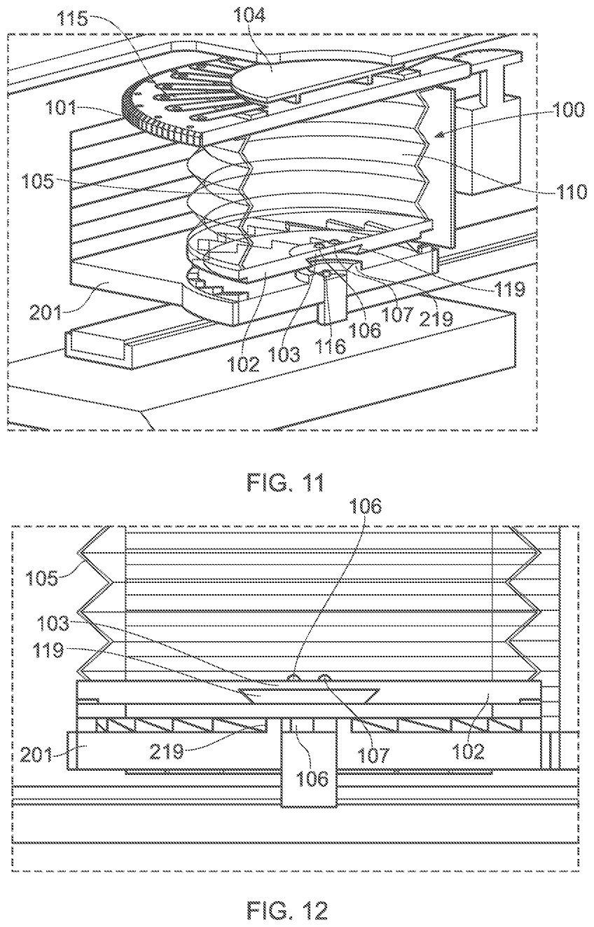

The cell processing unit 200 includes an agitator including an agitation plate 201, a base plate 202 and one or more linear actuators 203 which act upon a lower surface of the agitation plate 201 so as to raise and lower the agitation plate 201 relative to the bioreactor 100. The agitation mechanism may further include a pivotable rod 204 such that the agitation plate 201 can pivot about the pivotable rod 204. The bioreactor 100 may be preassembled to the agitation plate 201, or alternatively the agitation plate 201 is moved into contact with the base section 102 in order to assemble the bioreactor 100 to the agitation plate 201 (see FIG. 11). The bioreactor 100 may abut the agitation plate 201 or be coupled to the agitation plate 201.

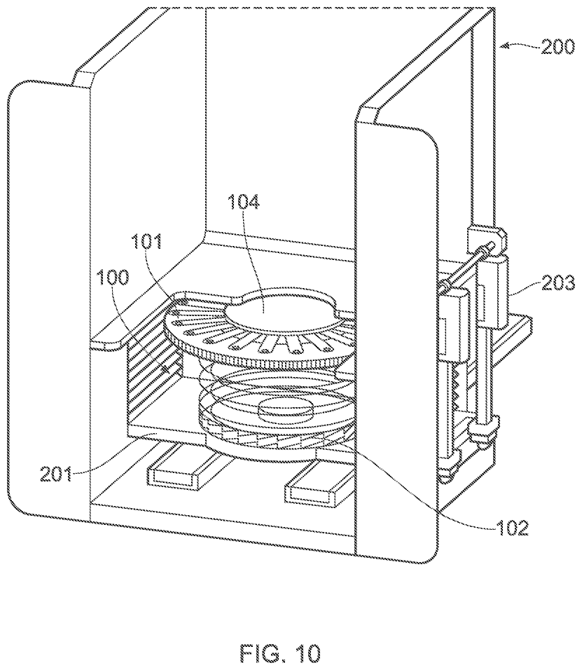

According to another example of the linear actuator as illustrated in FIG. 10, the linear actuator 203 includes a rail and a carriage. The carriage connects to an edge of the agitation plate 201 and the rail extends upwards toward the top section 101 of the bioreactor 100. Movement of the carriage along the rail raises and lowers the agitation plate 201.

FIGS. 11 and 12 show one example of a coupling between the base section 102 of the bioreactor 100 and the agitation plate 201. The base section 102 has an attachment feature 119 and the agitation plate 201 has a corresponding attachment feature 219. Such a coupling may provide an electrical connection between the sensors of the bioreactor base and a controller or the like of the cell processing unit.

Alternatively, as illustrated in FIG. 5, the base section 102 of the bioreactor 100 is affixed to a mounting piece 206. The mounting piece 206 and the agitation plate 201 each have corresponding fixation apertures which can receive a screw or other fixation means to mount the mounting piece 206 to the agitation plate 201.

FIGS. 4, 5 and 7 illustrate the optical sensors 116, 117 mounted in an aperture of the agitation plate 201. FIG. 7 illustrates an upstand 207 extending between the agitation plate 201 and a base plate 202. The upstand 207 positions the optical sensors 116, 117 close to the base section 102 of the bioreactor and includes an optical sensor mounting block 208. This mounting block 208 secures the optical sensors 116, 117 so that they are correctly pitched and aligned with the optical dots 106, 107.

In one example, as illustrates in FIGS. 6A and 6B, the upstand 207 is fixed and does not move with the agitation plate 201. Therefore, during agitation of the bioreactor, the optical sensors 116, 117 are not within range of the optical dots 106, 107, and when a measurement is taken, the agitation plate 201 is lowered to a position where the optical dots 106, 107 are within range of the optical sensors 116, 117.

In another example, the optical sensors 116, 117 are mounted to the agitation plate 201 so that the optical sensors 116, 117 move with the agitation plate 201 and remain stationary with respect to the optical dots 106, 107. In this arrangement, the optical sensors 116, 117 can continuously or periodically take measurements during agitation of the bioreactor.

The temperature sensor 121 may be connected to the agitation plate 201. In this way, the temperature sensor 121 may detect a temperature of the exterior of the bioreactor 100.

In use, the bioreactor 100 may be rotated. FIG. 7 illustrates a rotating electrical connection 209 (e.g., a slip ring) incorporated into the agitation plate 201 to provide an electrical connection between the cell processing unit 200 and the bioreactor 100. The rotating electrical connection 209 may provide a connection with the chemical sensor 120. The rotating electrical connection 209 provides an electrical connection while the bioreactor 100 is rotated.

The sensors, which may include the optical sensors 116, 117, chemical sensors 120 and/or temperature sensor 121, as described herein are coupled to a controller (not shown). The controller may be either separate to or integrated with the cell processing unit 200, and the sensors may be coupled to the controller via a wired or a wireless (e.g., Bluetooth) connection. The controller may be connected to a user interface to allow a user to monitor various parameters measured by the sensors, such as oxygen concentration, carbon dioxide concentration, pH, glucose, lactate, and/or temperature.

The cell processing unit 200 and the bioreactor 100 may be manually controlled in response to the measured parameters. For example, a user may alter the parameters of the cell processing unit 200 and/or the bioreactor 100 by adjusting the gas concentration or the temperature within the cell processing unit 200. The user may additionally or alternatively initiate actuation of the agitation plate 201 to mix the contents of the bioreactor 100. The user may additionally or alternatively connect accessories to the cell processing platform 104 to add cells, media, beads and/or viruses to the bioreactor, or the user may connect accessories to the cell processing platform 104 to extract waste media from the bioreactor or to extract a sample for further testing.

In examples, the controller may automatically control one or more operations of the cell processing unit 200 to change such parameters and thus adjust the condition within the bioreactor 100. For example, the controller may have pre-programmed target parameters, or alternatively a user can input the target parameters. The controller will control the operations of the cell processing unit 200 until the measured parameters are equal to the target parameters.

According to one example, the optical sensors 116, 117 direct light to the optical dots 106, 107. For example, the optical sensors 116, 117 have an LED that directs light onto an optical dot 106, 107. The optical dots 106, 107 emit a fluorescence in response to the incident light. This fluorescence response is then detected by the optical sensor 116, 117 as a fluorescence signal. The received fluorescence signal is associated with one or more of a dissolved oxygen parameter, a pH parameter and/or a dissolved carbon dioxide parameter. This sensed parameter relates to a condition within the bioreactor 100, such as the gas content, the dissolved gas content in the cell suspension, the cell suspension pH or the like. The sensed parameter is sent to the controller. The controller then controls the operation of the cell processing unit 200 to adjust the condition within the bioreactor 100 until the sensed parameter is equal to the target parameter.

The operation of the cell processing unit 200 may be controlled manually by a user or automatically by the controller to modify the flow rate or the concentration of oxygen entering the bioreactor 100 to adjust the gas concentration in the bioreactor 100. The cell processing unit 200 may alternatively or additionally be controlled to adjust the gas concentration in the cell processing unit 200, the gases in the cell processing unit 200 can equilibrate with the gases in the bioreactor 100 through a gas permeable material of the container 110 to adjust the gas concentration within the bioreactor. The cell processing unit 200 may alternatively or additionally be controlled to move the agitation plate 201 so that the base section 102 of the bioreactor moves toward the top section 101 of the bioreactor thereby compressing the sidewall 105 and stimulating mixing of the contents in the bioreactor. This mixing can increase the dissolved oxygen content in the cell suspension. The agitation plate 201 can also be controlled to provide compression, rocking, swirling and/or rotating of the bioreactor 100. Specific agitation parameters can be controlled, such as the rate of compressions of the bioreactor, the rocks of the bioreactor per time unit, the longitudinal displacement of the base section 102 with respect to the top section 101 (i.e., displacement during compression), and the like. The cell processing unit 200 can also be controlled to add new media into the bioreactor 100 and/or to remove waste media. The cell processing unit 200 can also be controlled to adjust the temperature within the cell processing unit 200, for example using a heater and/or cooler.

Other examples of parameters which can be manually controlled by a user or automatically controlled by a controller include, but are not limited to, the timing and volume of fresh media addition to the bioreactor, the timing and volume of culture media to be removed from the bioreactor, the timing and volume of a test sample to be taken from the bioreactor, the timing of the washing of cells in the bioreactor, the timing of the separation of cells in the bioreactor, the timing of the removal (harvesting) of cells from the bioreactor, and the like.

As illustrated in the figures, the container 110 is a bellows-based container. FIGS. 13 to 15 show the sidewall 105 of the container including a plurality of lateral rigid sections 301. The lateral rigid sections 301 are arranged in parallel with the base section 102. Each pair of adjacent lateral rigid sections 301 is interleaved with a deformable region 302 so as to allow compression of the bioreactor along the longitudinal axis. The deformable regions 302 may be hinges that alternate inward and outward to provide collapsibility of the container 110. The hinges may be formed by thinning of the sidewall 105 material, and directionality of the hinges may be provided by thinning on either the inner or the outer side of the sidewall 105. The lateral rigid sections 301 and deformable regions 302 extend from the top section 101 of the container to the base section 102 allowing for complete compression of the container 110.

The sidewall 105 may be formed from thermoplastic elastomer (TPE), silicone or low density polyethylene (LDPE), however the sidewall 105 may be formed from any suitable flexible material. The flexible material may be a biocompatible material. The sidewall 105 may be formed by injection molding or blow molding. The sidewall material may form the sidewall 105 and at least a portion, or all of, of the base section 102 which can be supported by a rigid portion of the base section 102 as discussed below. The sidewall material may be transparent, translucent or opaque.

The base section 102 of the container 110 illustrated in FIGS. 13 to 15 is a rigid base section 102. The base section may be a separate component, attached to the sidewall 105. The rigid base section 102 provides an optimal surface for cell adhesion and growth. The base section 102 may be formed from polycarbonate (PC) or high density polyethylene (HDPE), however the base may be formed from any suitable rigid material. The rigid material may be a biocompatible material. The base section material is preferably transparent or translucent (i.e., non-opaque), however the base section 102 may be opaque and have a transparent or translucent (i.e., non-opaque) sensor window 103. The base section 102 or sensor window 103 may be transparent to the light emitted by an LED of an optical sensor 116, 117, and is transparent to the fluorescence emitted by an optical dot 106, 107. The sensor window 103 may be formed by co-injection, however any other suitable method of integrally forming the sensor window 103 with the base section 102 may be used. The base section 102 and the sensor window 103 may alternatively be connected by a fluid-tight seal. The base section 102 may be formed by injection molding. The optical dots 106, 107 and/or the chemical sensor 120 may thus be integrally formed in the base section 102 during the injection molding process. For example, the sensor window 103 may be overmolded onto the optical dots 106, 107, or the sensor window 103 and the optical dots 106, 107 may be co-injected.

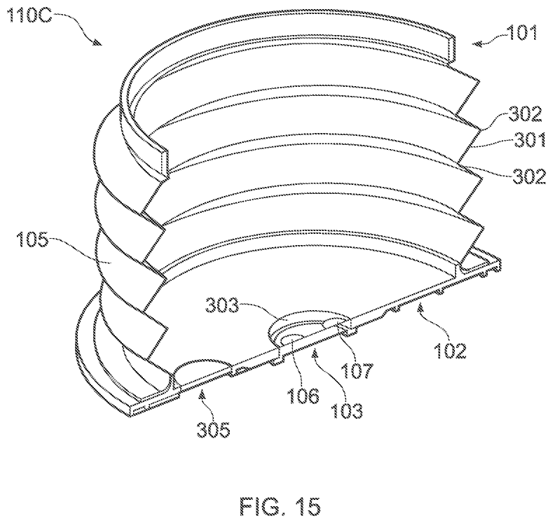

The geometry of the base section 102 may be modified to improve harvesting, for example, the base section 102 may be sloped or frustoconical, for example toward an outlet. As shown in FIG. 15, the base section 102 can further include a harvesting valve 305. The harvesting valve 305 may be a septum seal. The harvesting valve 305 may be formed from TPE. The harvesting valve 305 may be co-injected with the material of the base section.

The sidewall 105 is connected to the base section 102, for example by overmolding the components or by hot plate welding. Preferably, the sidewall 105 and the base section 102 are connected in a way that provides a smooth inner surface of the bellows container 110 to prevent cell trapping and fluid hang up. The sidewall 105 and base section 102 are connected such that the lowest deformable region 302 is directly adjacent to the base section 102. This allows for a more complete compression of the sidewall 105, thereby increasing the mixing capability of the bioreactor 100.

The base section 102 may be formed by a combination of an inner gas permeable material and an outer rigid, non-gas permeable material. The rigid material may comprise a plurality of openings, thereby allowing gas, including oxygen, to permeate into the cell suspension. The sidewall 105 may also be formed from the gas permeable material. The gas permeable material can be injection molded to form the sidewall 105 and a portion of the base section 102 which is supported by the rigid, non-gas permeable material. The gas permeable material may be silicone.

The following are exemplary embodiments of the bioreactor materials and manufacture:

### EXAMPLE 1

FIGS. 13A and 13B illustrate a first example of a bellows container 110A. The sidewall 105 is formed from an opaque TPE by injection molding the TPE to form the bellows structure. The rigid base section 102 is formed from a translucent PC by injection molding. A lower end portion of the TPE bellows wall is overmolded onto the PC base such that lowest deformable region 302 is directly adjacent to the base section 102.

As shown in FIG. 13A, the TPE bellows form only the sidewall 105 and an upper ridge suitable for connection with a cell processing platform or other lid.

Alternatively, as shown in FIG. 13B, the TPE bellows form the sidewall 105 and a portion of the base section 102 which is supported by the rigid PC base. As the TPE is opaque, an opening 303 is formed at a central portion of the TPE base such that the underlying PC base provides a transparent or translucent sensor window 103.

### EXAMPLE 2

A second example of the bellows container is constructed in the same way as the first example bellows container (Example 1), however the sidewall 105 is formed from silicone. The silicone sidewall may be opaque or translucent.

### EXAMPLE 3

FIG. 14 illustrates a third example of the bellows container 110B. The sidewall 105 is formed from a translucent TPE by injection molding the TPE to form the bellows structure. The rigid base section 102 is formed from co-injected HDPE and PC such that a portion of the base section 102 is formed from an opaque HDPE, and a translucent PC sensor window 103 is formed at a central portion of the base section 102. A lower end portion of the TPE bellows wall is overmolded onto the HDPE base such that lowest deformable region 302 is directly adjacent to the base section 102.

### EXAMPLE 4

FIG. 15 illustrates a fourth example of the bellows container 110C. The sidewall 105 is formed from a translucent LDPE by blow molding the LDPE to form the bellows structure. The rigid base section 102 is formed from co-injected HDPE and PC such that a portion of the base section 102 is formed from an opaque HDPE, and a translucent PC sensor window 103 is formed at a central portion of the base section 102.

A lower end portion of the TPE sidewall 105 is connected to the HDPE base section 102 by hot plate welding the lower end portion of the TPE bellows to the perimeter of the base such that lowest deformable region 302 of the TPE sidewall is directly adjacent to the base section 102.

1. A bioreactor for cell processing, comprising:
a container including
a base section comprising a sensor window chosen from a transparent sensor window or a translucent sensor window,
a top section arranged opposite to the base section and comprising a fluid inlet and a fluid outlet, and
a sidewall extending between the base section and the top section and defining an internal volume of the container adapted to hold a cell suspension; and
at least one optical element disposed on the sensor window within the internal volume, the at least one optical element adapted to emit a fluorescence signal in response to incident light, the fluorescence signal associated with one or more parameters of the cell suspension.

2. The bioreactor according to claim 1, further comprising at least one optical sensor positioned proximate to an outer surface of the sensor window.

3. The bioreactor according to claim 2, wherein the at least one optical sensor is in alignment with the at least one optical element.

4. The bioreactor according to claim 2, wherein the at least one optical sensor comprises an Light Emitting Diode (LED) arranged to emit light onto the at least one optical element, and wherein the at least one optical sensor is configured to receive the fluorescence signal emitted by the at least one optical element.

5. (canceled)

6. (canceled)

7. The bioreactor according to claim 1, wherein the at least one optical element is positioned at a position chosen from at or near a center position of the base section.

8. The bioreactor according to claim 1, wherein the bioreactor further comprises at least one chemical sensor.

9. The bioreactor according to claim 8, wherein the at least one chemical sensor includes at least one sensor chosen from a glucose sensor, a lactate sensor, or an enzymatic-based sensor.

10. (canceled)

11. The bioreactor according to claim 1, wherein the bioreactor further comprises a temperature sensor.

12. The bioreactor according to claim 1, wherein the sidewall of the container comprises a compressible wall element.

13. The bioreactor according to claim 12, wherein the base section is connectable to an agitator operable to move the base section relative to the top section to compress or extend the compressible wall element.

14. A cell processing system comprising:
a bioreactor according to claim 1,
an optical sensor positioned proximate to an outer surface of the sensor window to sense a fluorescence signal emitted by the at least one optical element associated with one or more parameters of the cell suspension, and
a controller configured to receive a sensor signal from the optical sensor, the signal corresponding to the one or more parameters of the cell suspension.

15. The cell processing system according to claim 14, further comprising an agitator arranged to engage the base section of the bioreactor and adapted to move the base section.

16. The cell processing system according to claim 15, wherein the agitator comprises an agitation plate arranged to engage the base section of the container, and wherein the agitation plate includes an aperture for receiving the optical sensor.

17. The cell processing system according to claim 15, wherein the sidewall of the container comprises a compressible wall element, and wherein the controller is configured to control the agitator to move the base section relative to the top section to stimulate mixing of a fluid within the bioreactor.

18. The cell processing system according to claim 14, wherein the controller is configured to adjust a condition within the bioreactor based on the received sensor signal.

19. The cell processing system according to claim 18, wherein the controller is configured to adjust the condition within the bioreactor until a parameter is equal to a target parameter.

20. The cell processing system according to claim 18, wherein the controller is configured to adjust the condition within the bioreactor by adjusting the gas flow into the bioreactor.

21. A method of cell processing, the method comprising:
providing a cell processing system comprising: a container including a base section comprising a sensor window chosen from a transparent sensor window or a translucent sensor window, a top section arranged opposite to the base section and comprising a fluid inlet and a fluid outlet, and a sidewall extending between the base section and the top section and defining an internal volume of the container adapted to hold a cell suspension; at least one optical element disposed on the sensor window within the internal volume, the at least one optical element adapted to emit a fluorescence signal in response to incident light, the fluorescence signal associated with one or more parameters of the cell suspension; and an agitator arranged to engage the base section of the bioreactor and adapted to move the base section; and
sensing a fluorescence signal emitted by the optical element associated with the one or more parameters of the cell suspension using the optical sensor.

22. The method according to claim 21, further comprising adjusting a condition within the bioreactor based on the sensed fluorescence signal.

23. The method according to claim 22, wherein adjusting the condition within the bioreactor includes adjusting the gas flow into the bioreactor.

24. The method according to claim 22, wherein the sidewall of the container comprises a compressible wall element, and wherein adjusting the condition within the bioreactor includes moving the base section relative to the top section to stimulate mixing of a fluid within the bioreactor.
